(19) United States
(12) Patent Application Publication
(10) Pub. No.: US 2017/0290916 A1
(43) Pub. Date: Oct. 12, 2017

(54) Materials and Methods for the Delivery of a Nanocarrier to the Brain

(71) Applicants: Ajeet KAUSHIK, Doral, FL (US); Madhavan NAIR, Coral Gables, FL (US)

(72) Inventors: Ajeet KAUSHIK, Doral, FL (US); Madhavan NAIR, Coral Gables, FL (US)

(73) Assignee: The Florida International University Board of Trustees, Miami, FL (US)

(21) Appl. No.: 15/481,689

(22) Filed: Apr. 7, 2017

Related U.S. Application Data
(60) Provisional application No. 62/319,656, filed on Apr. 7, 2016.

(51) Int. Cl. A61K 47/00; A61K 47/02

(52) U.S. Cl. CPC A61K 47/00; A61K 47/02; A61K 47/48953; B82Y 5/00

(57) ABSTRACT

Materials and methods for magnetically guided delivery of nanoparticles across the blood brain barrier (BBB) in the central nervous system (CNS) are provided. The method can comprise injecting a subject with an aqueous solution comprising magneto-electro nanoparticles and applying a static magnetic field directed toward the subject brain, thereby inducing a stimulus response of the nanoparticles in a controlled manner. Materials and methods provided herein are effective in delivering the nanoparticles across the BBB in the brains of animal subjects including mice and non-human primate such as baboon.

### CROSS-REFERENCE TO A RELATED APPLICATION

This application claims the benefit of U.S. provisional application Ser. No. 62/319,656, filed Apr. 7, 2016, which is incorporated herein by reference in its entirety.

### GOVERNMENT SUPPORT

This invention was made with government support under Grant Nos. RO1DA042706, R01-DA040537, RO1-DA037838, and 1R21-MH101025 awarded by the National Institutes of Health. The government has certain rights in the invention.

### BACKGROUND OF INVENTION

Many therapeutic carriers are limited in their effectiveness in treating disorders affecting the brain because these carriers cannot pass the blood brain barrier (BBB) (Pardridge, W. M. Drug and gene targeting to the brain with molecular Trojan horses. Nat. Rev. Drug Dis. 1, 131 (2002); Pardridge, W. M. Molecular Trojan horses for blood-brain barrier drug delivery. Current Opin. Pharmacol. 6, 494 (2006); Pardridge, W. M. Blood-brain barrier delivery. Drug Dis. Today 12, 54 (2007); Löscher, W. & Potschka, H. Drug resistance in brain diseases and the role of drug efflux transporters. Nat. Rev. Neurosci. 6, 591-602 (2005)). The BBB facilitates and controls homeostasis in the brain by relying on tight junctions between endothelial cells, feet of astrocytes, and pericytes, effectively blocking drug molecules to pass through (Nowacek, A. & Gendelman, H. E. NanoART, neuroAIDS and CNS drug delivery. Nanomedicine 4, 557-574 (2009); Lavan, D. A., McGuire, T. & Langer, R. Small-scale systems for in vivo drug delivery. Nat. Biotech. 21, 1184-1191 (2003)).

With increased development in imaging tools for diagnosis and disease monitoring, nano-formulations (NFs) comprising nanoscopic carriers loaded with drugs of interests have attracted much attention due to the possibility of achieving site-specific delivery and stimulus-responsive release mechanisms (Mikula, S., Binding, J. & Denk, W. Staining and embedding the whole mouse brain for electron microscopy. Nat. Methods. 9, 1198 (2012); Wegscheid, M. L., Morshed, R. A., Cheng, Y. & Lesniak, M. S. The art of attraction: applications of multifunctional magnetic nanomaterials for malignant glioma. Exp. Opin. Drug Del. 11, 957. (2014); Cheng, Y., Morshed, R. A., Auffinger, B., Tobias, A. L. & Lesniak, M. S. Multifunctional nanoparticles for brain tumor imaging and therapy. Adv. Drug Del. Rev. 66, 42 (2014); Cheng, Y. et al. Blood-Brain Barrier Permeable Gold Nanoparticles: An Efficient Delivery Platform for Enhanced Malignant Glioma Therapy and Imaging. Small 10, 5137 (2014); Liu, L. et al. Polymeric micelles anchored with TAT for delivery of antibiotics across the blood-brain barrier. Pept. Sci. 90, 617 (2008); Santra, S. Yang, H. Holloway, P. H. Stanley, J. T. & Mericle, R. A. Synthesis of water-dispersible fluorescent, radio-opaque, and paramagnetic CdS: Mn/ZnS quantum dots: a multifunctional probe for bioimaging. J. Am. Chem. Soc. 127, 1656 (2005)). One major limitation of these methods is a general lack of control over their release mechanisms. When such mechanisms are not controlled, carriers can become entrapped in an endosomal pathway and fail to carry a drug to the target sites, or the drug can become exocytosed. Further, larger NFs measuring above 100 nm in size can be directly filtered out by the reticuloendothelial system organs prior to their navigation across the BBB (Pardridge, W. M. Drug and gene targeting to the brain with molecular Trojan horses. Nat. Rev. Drug Dis. 1, 131 (2002); Pardridge, W. M. Molecular Trojan horses for blood-brain barrier drug delivery. Current Opin. Pharmacol. 6, 494 (2006); Pardridge, W. M. Blood-brain barrier delivery. Drug Dis. Today 12, 54 (2007); Bourzac, K. Nanotechnology: Carrying drugs. Nature 491, S58-S60 (2012)).

Recently, various approaches have been introduced in hope of increasing the efficacy of delivering NFs across the BBB (Kaushik, A., Jayant, R. D., Sagar, V. & Nair, M. The potential of magneto-electric nanocarriers for drug delivery. Exp. Opin. Drug Del. 11, 1635 (2014); Sagar, V. Pilakka-Kanthikeel, S. Pottathil, R. Saxena, S. K. & Nair, M. Towards nanomedicines for neuroAIDS. Rev. Med. Virol. 24, 103 (2014); Mikula, S., Binding, J. & Denk, W. Staining and embedding the whole mouse brain for electron microscopy. Nat. Methods. 9, 1198 (2012); Wegscheid, M. L., Morshed, R. A., Cheng, Y. & Lesniak, M. S. The art of attraction: applications of multifunctional magnetic nanomaterials for malignant glioma. Exp. Opin. Drug Del. 11, 957. (2014); Cheng, Y., Morshed, R. A., Auffinger, B., Tobias, A. L. & Lesniak, M. S. Multifunctional nanoparticles for brain tumor imaging and therapy. Adv. Drug Del. Rev. 66, 42 (2014)). However, these approaches are not well adopted because they offer transient BBB openings and exhibit side effects such as CNS cell damage and delayed recovery. For example, delivery of nanostructures comprising polymers and metals mediated by the trans-activator of transcription (TAT) protein across the BBB has been demonstrated to lack the control needed to deliver NFs in an on-demand fashion (Cheng, Y. et al. Blood-Brain Barrier Permeable Gold Nanoparticles: An Efficient Delivery Platform for Enhanced Malignant Glioma Therapy and Imaging. Small 10, 5137 (2014); Liu, L. et al. Polymeric micelles anchored with TAT for delivery of antibiotics across the blood-brain barrier. Pept. Sci. 90, 617 (2008); Santra, S. Yang, H. Holloway, P. H. Stanley, J. T. & Mericle, R. A. Synthesis of water-dispersible fluorescent, radio-opaque, and paramagnetic CdS: Mn/ZnS quantum dots: a multifunctional probe for bioimaging. J. Am. Chem. Soc. 127, 1656 (2005)).

Therefore, there remains a critical need to develop an effective nanocarrier that can be externally guided for on-demand delivery of carriers across the BBB without compromising the drugs' efficacy for treating CNS diseases.

### BRIEF SUMMARY

The subject invention provides materials and methods for magnetically-guided delivery of nanoparticles across the blood brain barrier (BBB).

In one embodiment, the method comprises injecting a subject with an aqueous solution comprising a plurality of magneto-electro nanoparticles and applying an alternating current (AC) magnetic field directed toward the subject brain, thereby inducing a stimulus response of the nanoparticles in a controlled manner. In exemplary embodiments, the materials and methods provided herein are effective in delivering the nanoparticles across the BBB in the brains of mammals.

According to some embodiments of the subject invention, the nanoparticles comprise ferromagnetic and multiferroic materials having a core-shell structure. Nanoparticles comprising magneto-electro materials are referred to herein as magneto-electro nanocarriers (MENCs).

In preferred embodiments, each MENC is loaded with a therapeutic agent. The agent may be capable of, for example, treating diseases affecting the central nervous system (CNS) including, but not limited to, brain tumors, cancer, neuroAIDS, and other neurodegenerative disorders.

Optionally, each MENC can be encapsulated in a coating layer comprising one or more of biocompatible polymers.

In preferred embodiments, the magnetic stimulation needed to trigger the delivery of therapeutic agents carried by the MENCs comprises a low-energy AC magnetic field capable of inducing an electric dipole moment within each MENC. The formation of the electric dipole moment can in turn cause an on-demand stimulus response of the MENCs to release the therapeutic agents attached thereto at a target treatment area in the brain.

Advantageously, materials and methods provided herein have demonstrated non-toxic and non-invasive delivery of crystalline MENCs across the BBB of a subject brain in-vivo while retaining the nanocarriers' (and any therapeutic agents attached thereto) structural and chemical integrity, affording the ability to provide controlled and on-demand delivery of otherwise BBB-impenetrable therapeutic agents to treat a wide range of CNS-related diseases.

### BRIEF DESCRIPTION OF THE DRAWINGS

FIGS. 1A-1D are transmission electron microscopy (TEM) images of an embodiment of the MENCs, and of their atomic planes with respect to BTO (BaTiO₃; FIG. 1B) and CFO (CoFe₂O₄; FIGS. 1C-1D).

FIG. 2A-2C show the characterization results of CoFe₂O₄ and the exemplary MENCs (BaTiO₃@CoFe₂O₄). FIG. 2A shows the result of a vibrating sample magnetometry (VSM) study of CoFe₂O₄ and the MENCs (BaTiO₃@CoFe₂O₄). FIG. 2B is an X-ray diffraction pattern that demonstrates the phase purity and crystallinity of the MENCs. FIG. 2C is a Raman spectrum that explains the functionality of the exemplary MENCs.

FIGS. 3A-3C show the results of an in-vitro toxicity evaluation of exemplary MENCs using MT assay at various proposed doses with respect to an average mice weight of 20±5 g. An optimized dose of 10 mg/kg MENCs corresponding to 0.25 mg/mL was injected in mice. For MENCs injection, the mouse was under anesthesia and after injection the physical condition of each mouse was under continuous monitoring. FIG. 3A shows the MTT assay results based on primary human astrocytes. FIG. 3B shows the MTT assay results based on SKMNCs. FIG. 3C is an image of a mouse subject on an injection bed. Significance was considered to be p<0.05.

FIGS. 4A-4B show two in-situ TEM images of a mouse's brain tissue without the injection of exemplary MENCs, i.e. the control subject. In FIG. 4A, the arrow indicates the direction of MENCs' movement across tight junctions of endothelial (E) cell layer, whereas (A) indicates astrocytes. In both figures, the dotted circles represent the nuclei of the cells. Scale bars: 1 μm (FIG. 4A) and 0.5 μm (FIG. 4B).

FIGS. 5A-5F show a set of in-situ TEM images of the mouse's brain tissue after injection of exemplary MENCs. MENCs were capable of crossing the BBB (e.g., see FIG. 5A in comparison with FIG. 4A), direction of movement across tight junctions of endothelial (E) cells layer is indicated by arrows in FIGS. 5A and 5C. MENCs were able to reach target sites, including neurons (N), astrocytes (A), and microglia (M), and were also observed in smooth muscle cell (S), endothelial cells (E) and blood cells (¤). Most MENCs were uniformly distributed in brain tissue/cells and were able to reach nucleus, as indicated by dotted circles in FIGS. 5A, 5C, and 5E, but some agglomeration of MENCs in cell membranes and their entrapment in endosomes was also observed, indicated by solid arrow heads in FIGS. 5B and 5C. “*” represents synapses in FIG. 5C, J represents neuromuscular junction between S and at axon terminal in FIG. 5E. A layer of Schwann cells (sc) surrounding it is also observed in FIG. 5C. Scale bars: 1 μm (FIGS. 5A, 5B, and 5E) and 0.5 μm (FIGS. 5C, 5D, and 5F).

FIGS. 6A-6K show the in-situ STEM results, confirming the elemental analysis and distribution of exemplary MENCs inside the mouse's brain tissue following the MENCs treatment. Various spots ware selected for both convergent beam electron diffraction (CBED) and energy-dispersive spectroscopy (EDS) measurements. Each spot, highlighted by a solid circle, was analyzed morphologically to understand MENCs distribution in brain cells. STEM-based CBED pattern (FIGS. 6A-6F) were analyzed for the evaluation of MENCs crystallinity in the brain tissue (FIG. 6J), the zone axis is in the [011] direction, and EDS spectra obtained from FIGS. 6G-6I for elemental analysis of MENCs in brain tissue samples (FIG. 6K).

FIGS. 7A-7D show the results of histopathology of phosphate buffered saline (PBS)-injected control (Ct) and MENCs-injected mice (NpT) with respect to the kidney (FIG. 7A), liver (FIG. 7B), spleen (FIG. 7C), and brain (FIG. 7D) of the mice (n=3). Histopathological analysis confirmed no observation of organ tissues damage due to the presence of MENPs in mice, proving the biocompatibility of MENPs for biological applications.

FIG. 8 is a blood toxicity analysis of MENCs-injected mice (NpT) at day 2 and 7 (n=3). The average values of various parameters of liver and renal function were studied and compared with a reference value. Blood toxicity profile showed no toxicity at 10 mg/kg of MENCs (n=3).

FIG. 9A-9C show the results of sensorimotor activities after day 2 and 7 following injection of exemplary MENCs. Indicated treatments are: Control (no injection), PBS (saline, I.V.) and MENCs (10 mg/kg, I.V.). Mice tested after day 2 were re-tested at day 7 and the results from each day are shown. FIG. 9A shows the results of the grip strength test. The average forelimb grip strength of each animal was normalized to its corresponding body weight. FIG. 9B shows the results of the horizontal bar test. Each score represents the combined duration of a mouse on the 2 mm and 4 mm bars. FIG. 9C shows the results of the accelerating rotarod test. The time (in seconds) represents the latency to fall and the time each mouse spent on the rotating rod. All data are presented as the mean±the standard error of the mean (S.E.M.). The results were analyzed within group comparisons (days 2 and 7) using t-tests and across group (Control, PBS, and MENCs) comparisons for each day using one-way analysis of variance (ANOVA) followed by Tukey's post-hoc tests to determine statistical significance (Graph Pad 5 Software, Inc., La Jolla, Calif., USA). Significance was considered to be p<0.05.

FIG. 10A depicts the magnetic resonance imaging (MRI) phantom study results showing changes in contrast as a function of the concentration of MENPs, while FIG. 10B shows the relationship between the concentration of MENPs and changes in T₂ intensity.

FIG. 11A shows ex-vivo MRI images of the brain and the liver of MENPs-injected mice as a function of static magnetic field exposure time, while FIG. 11B shows changes in T₂ contrast of the brain and the liver of MENPs-injected mice as a function of time. The T₂ value saturated at 3 hrs of magnetic exposure, confirming it as the optimized time to achieve delivery to the brain.

FIG. 12 depicts the experimental setup for an MRI-guided MENC delivery to the central nervous system (CNS) of a baboon subject.

FIG. 13 illustrates brain MRI images of the MENCs-injected baboon subject. Reduction in contrast confirms the presence of MENCs in the brain of the subject.

FIG. 14 shows abdominal MRI images of the MENCs-injected baboon subject. Reduction in contrast confirms the presence of MENCs in the periphery system of the subject.

FIGS. 15A-15C demonstrate pathological staining of the liver, lung, and bladder, respectively, of the baboon subject.

FIG. 16 demonstrates pathological staining of the kidney of the baboon subject.

FIGS. 17A-17B demonstrate pathological staining of the heart and adrenal gland, respectively, of the baboon subject.

FIGS. 18A-18C demonstrate pathological staining of the spleen, stomach, and uterus, respectively, of the baboon subject.

FIGS. 19A-19B demonstrate pathological staining of the rolled intestine and the intestine lymphoplasmacytic chronic active entries, respectively, of the baboon subject.

FIGS. 20A-20C demonstrate pathological staining of the intestine (GALT), intestine small (GALT, mild inflammation), and intestine large, respectively, of the baboon subject.

FIGS. 21A-21C demonstrate pathological staining of the cortex, mid brain, and cerebellum, respectively, of the baboon subject.

### DETAILED DISCLOSURE

The subject invention provides materials and methods for magnetically-guided delivery of nanoparticles across the blood brain barrier (BBB). In some embodiments, the method comprises injecting a subject with an aqueous solution comprising a plurality of magneto-electro nanoparticles and applying an alternating current (AC) magnetic field directed toward the brain, thereby inducing a stimulus response of the nanoparticles in a controlled manner. In exemplary embodiments, the materials and methods provided herein are effective in delivering the nanoparticles across the BBB in the brains of a mammal.

Advantageously, materials and methods provided herein have demonstrated non-toxic and non-invasive delivery of crystalline MENCs across the BBB of a subject brain in-vivo while retaining the nanocarriers' (and any therapeutic agents attached thereto) structural and chemical integrity, thereby facilitating controlled and on-demand delivery of otherwise BBB-impenetrable therapeutic agents treat a wide range of CNS-related diseases.

According to some embodiments of the subject invention, the nanoparticles comprise ferromagnetic and multiferroic materials having a core-shell structure. Nanoparticles comprising magneto-electro materials are referred to herein as magneto-electro nanocarriers (MENCs).

In preferred embodiments, each MENC is loaded with a therapeutic agent capable of treating diseases affecting the central nervous system (CNS) including, but not limited to, brain tumors, cancer, neuroAIDS, and other neurodegenerative disorders.

Optionally, each MENC can be encapsulated in a coating layer comprising one or more of biocompatible polymers.

MENCs according to the subject invention have a non-zero magnetic moment and, therefore, can be controlled remotely via application of an external magnetic field.

Advantageously, and unlike conventional magnetic nanoparticles (MNs), MENCs offer energy-efficient control of the intrinsic electric fields within the nanoparticles by an external magnetic field. This capability is a result of magneto-electric (ME) coupling in this class of nanostructures, even at body temperature. As a result, MENCs introduced in a biological microenvironment act as localized magnetic-to-electric-field nano-converters that allow remote control and generation of electric signals that affect the intrinsic molecular interactions within each MENC.

Exemplary MENCs comprise one or more of the following materials: iron, cobalt, zinc, cadmium, nickel, gadolinium, chromium, copper, manganese, terbium, europium, gold, silver, platinum, oxides of any of the preceding, alloys of any of the preceding, or mixtures thereof. Specific examples of MENCs include, but are not limited to, iron oxide, superparamagnetic iron oxide, Fe₃O₄, Fe₂O₄, FeₓPt_{y}, CoₓPt_{y}, MnFeₓO_{y}, CoFeₓO_{y}, NiFeₓO_{y}, CuFeₓO_{y}, ZnFeₓO_{y}, and CdFeₓO_{y}, wherein values of x and y vary depending upon the method of synthesis. In preferred embodiments, the MENCs comprise BaTiO₃@CoFe₂O₄, wherein BaTiO₃ is deposited onto CoFe₂O₄ surface in a core-shell structure.

In some embodiments, each MENC has a diameter smaller than approximately 50 nm, smaller than about 40 nm, smaller than about 35 nm, smaller than about 30 nm, smaller than about 25 nm, smaller than about 20 nm, smaller than about 15 nm, or smaller than about 10 nm. In some embodiments, the MENCs have sizes in the range of about 15 to about 20 nm, in the range of about 10 to about 15 nm, in the range of about 20 to about 25 nm, in the range of about 10 to about 50 nm, in the range of about 20 to about 50 nm, in the range of about 20 to about 40 nm, or in the range of about 10 to about 30 nm. Preferably, the MENCs have sizes in the range of about 20 to about 30 nm. The MENCs are small enough to penetrate the BBB and move into selected treatment areas.

In some embodiments, the MENCs are suspended in an aqueous diluent and injected into the bloodstream of a subject. The term “subject” as used herein, means a human, or a non-human mammal (e.g., a mouse, rat, dog, cat, cow, sheep, pig, or goat), including non-human primates (e.g., a baboon, monkey, or macaque).

In some embodiments, the concentration of MENCs in solution is between about 5 mg/kg to about 25 mg/kg, between about 6.5 mg/kg and about 20.3 mg/kg, or between about 8 mg/kg and about 12 mg/kg in human dosage. In an exemplary embodiment, the concentration of MENCs is about 10 mg/kg. When animal subjects are employed, the concentration of MENCs can be adjusted using appropriate interspecies scaling factors.

The aqueous diluent can be any of those that are known to a person skilled in this field and include, for example, distilled water, de-ionized water, pure or ultrapure water, saline, phosphate-buffered saline (PBS), and solutions containing other buffers that are compatible with the other components of the materials and methods provided herein.

The administration of the MENCs solution can be carried out generally in any desired manner or on any desired route of administration in order to achieve entry into the subject and transportation thereby to the targeted area (such as the BBB). Administration can be, for example, intravenous, via oral, subcutaneous, intramuscular, intranasal, pulmonal, or rectal route. In preferred embodiments, the MENCs solution is injected intravenously.

In some embodiments, the coating layer comprises one or more of biocompatible polymers such as glycerol monooleate (GMO), polyethylene glycol (PEG), and poly-L-lysine (PLL).

In some embodiments, the MENCs can be further modified to include a chemical tagging agent. The chemical tagging agent can be used to target the MENCs to the site of interest in the subject brain prior to application of the magnetic field. Examples include, but are not limited to, targeting antibodies, aptamers, and antigens, such as cancer antigens. The chemical tagging agent can be attached to the surface of the MENCs via, for example, an ionic or covalent bond.

In some embodiments, the therapeutic or diagnostic agent, or “drug,” delivered in the methods provided herein can be any drug capable of forming chemical bonds with the MENCs. In preferred embodiments, the drug is one that is useful in detecting and/or treating CNS diseases including, but not limited to, brain tumors, cancer, Alzheimer's disease, Parkinson's disease, Huntington's disease, neuroAIDS, and other neurodegenerative disorders. In preferred embodiments, the drug can have an ionic moiety to form an ionic bond with the MENCs, e.g. a carboxylic acid, a phosphate, a sulfonate, and/or an amine group.

Non-limiting examples of drugs include natural enzymes, proteins derived from natural sources, recombinant proteins, natural peptides, synthetic peptides, cyclic peptides, antibodies, cytotoxic agents, immunoglobins, beta-adrenergic blocking agents, calcium channel blockers, coronary vasodilators, cardiac glycosides, antiarrhythmics, cardiac sympathomimetics, angiotensin converting enzyme (ACE) inhibitors, diuretics, inotropes, cholesterol and triglyceride reducers, bile acid sequestrants, fibrates, 3-hydroxy-3-methylgluteryl (HMG)-CoA reductase inhibitors, niacin derivatives, antiadrenergic agents, alpha-adrenergic blocking agents, centrally acting antiadrenergic agents, vasodilators, potassium-sparing agents, thiazides and related agents, angiotensin II receptor antagonists, peripheral vasodilators, antiandrogens, estrogens, antibiotics, retinoids, insulins and analogs, alpha-glucosidase inhibitors, biguanides, meglitinides, sulfonylureas, thiazolidinediones, androgens, progestogens, bone metabolism regulators, anterior pituitary hormones, hypothalamic hormones, posterior pituitary hormones, gonadotropins, gonadotropin-releasing hormone antagonists, ovulation stimulants, selective estrogen receptor modulators, antithyroid agents, thyroid hormones, bulk forming agents, laxatives, antiperistaltics, flora modifiers, intestinal adsorbents, intestinal anti-infectives, antianorexic, anticachexic, antibulimics, appetite suppressants, antiobesity agents, antacids, upper gastrointestinal tract agents, anticholinergic agents, aminosalicylic acid derivatives, biological response modifiers, corticosteroids, antispasmodics, 5-HT4 partial agonists, antihistamines, cannabinoids, dopamine antagonists, serotonin antagonists, cytoprotectives, histamine H2-receptor antagonists, mucosal protective agent, proton pump inhibitors, H. pylori eradication therapy, erythropoieses stimulants, hematopoietic agents, anemia agents, heparins, antifibrinolytics, hemostatics, blood coagulation factors, adenosine diphosphate inhibitors, glycoprotein receptor inhibitors, fibrinogen-platelet binding inhibitors, thromboxane-A2 inhibitors, plasminogen activators, antithrombotic agents, glucocorticoids, mineralcorticoids, corticosteroids, selective immunosuppressive agents, antifungals, drugs involved in prophylactic therapy, AIDS-associated infections, cytomegalovirus, non-nucleoside reverse transcriptase inhibitors, nucleoside analog reverse transcriptse inhibitors, protease inhibitors, anemia, Kaposi's sarcoma, aminoglycosides, carbapenems, cephalosporins, glycopeptides, lincosamides, macrolies, oxazolidinones, penicillins, streptogramins, sulfonamides, trimethoprim and derivatives, tetracyclines, anthelmintics, amebicides, biguanides, cinchona alkaloids, folic acid antagonists, quinoline derivatives, Pneumocystis carinii therapy, hydrazides, imidazoles, triazoles, nitroimidzaoles, cyclic amines, neuraminidase inhibitors, nucleosides, phosphate binders, cholinesterase inhibitors, adjunctive therapy, barbiturates and derivatives, benzodiazepines, gamma aminobutyric acid derivatives, hydantoin derivatives, iminostilbene derivatives, succinimide derivatives, anticonvulsants, ergot alkaloids, antimigrane preparations, biological response modifiers, carbamic acid eaters, tricyclic derivatives, depolarizing agents, nondepolarizing agents, neuromuscular paralytic agents, CNS stimulants, dopaminergic reagents, monoamine oxidase inhibitors, COMT inhibitors, alkyl sulphonates, ethylenimines, imidazotetrazines, nitrogen mustard analogs, nitrosoureas, platinum-containing compounds, antimetabolites, purine analogs, pyrimidine analogs, urea derivatives, anthracyclines, actinomycins, camptothecin derivatives, epipodophyllotoxins, taxanes, vinca alkaloids and analogs, antiandrogens, antiestrogens, nonsteroidal aromatase inhibitors, protein kinase inhibitor antineoplastics, azaspirodecanedione derivatives, anxiolytics, stimulants, monoamine reuptake inhibitors, selective serotonin reuptake inhibitors, antidepressants, benzisooxazole derivatives, butyrophenone derivatives, dibenzodiazepine derivatives, dibenzothiazepine derivatives, diphenylbutylpiperidine derivatives, phenothiazines, thienobenzodiazepine derivatives, thioxanthene derivatives, allergenic extracts, nonsteroidal agents, leukotriene receptor antagonists, xanthines, endothelin receptor antagonist, prostaglandins, lung surfactants, mucolytics, antimitotics, uricosurics, xanthine oxidase inhibitors, phosphodiesterase inhibitors, metheamine salts, nitrofuran derivatives, quinolones, smooth muscle relaxants, parasympathomimetic agents, halogenated hydrocarbons, esters of amino benzoic acid, amides (e.g. lidocaine, articaine hydrochloride, bupivacaine hydrochloride), antipyretics, hynotics and sedatives, cyclopyrrolones, pyrazolopyrimidines, nonsteroidal anti-inflammatory drugs, opioids, para-aminophenol derivatives, alcohol dehydrogenase inhibitor, heparin antagonists, adsorbents, emetics, opioid antagonists, cholinesterase reactivators, nicotine replacement therapy, vitamin A analogs and antagonists, vitamin B analogs and antagonists, vitamin C analogs and antagonists, vitamin D analogs and antagonists, vitamin E analogs and antagonists, and vitamin K analogs and antagonists.

In some embodiments, MENCs, due to their high magnetic moments, can be magnetically directed to a target treatment area by remotely applying an alternating current (AC) and/or direct current (DC) magnetic field. MENCs have a non-zero magneto-electricity; therefore, unlike their magnetic nanoparticles counterparts, the applied magnetic field can induce an electric dipole moment within each MENC. Changes in electric dipole moment can in turn weaken and subsequently break the chemical bonds between the MENC and any drug molecule bonded thereto when sufficient magnetic stimulation is reached, leading to a dissipation-free release mechanism with externally controllable outcome. In some embodiments, DC magnetic field is first directed toward the subject brain to guide the delivery of the MENCs to a target treatment area, and an AC magnetic field is then applied to facilitate the on-demand release of drug molecules to the target treatment area.

In some embodiments, only a low-energy external magnetic field, e.g. AC, is required to stimulate brain activity at any depth in the brain and release a therapeutic drug on-demand. The external magnetic field generated by an electromagnetic coil, for example, can be focused to act upon the MENCs in any particular region of the brain needing the treatment. The strength of the magnetic field applied can be, for example, at least 10 Oe or at least 15 Oe. In various cases, the strength is about 20 to about 45 Oe, about 30 to about 35 Oe, or about 45 to about 65 Oe.

The magnetic field can have a frequency of about 10 Hz to about 100 Hz, or about 500 Hz to about 1000 Hz.

The methods disclosed herein provide a tailorable way to deliver the drug of interest to the subject. Choice of strength of the magnetic field and length of time the field is applied allows for a predetermined amount of drug to be released from the MENCs. In some embodiments, the applied magnetic field for delivery across the BBB is less than about 1 T, and can be, for example, about 0.2 T, about 0.5 T, or about 0.9 T. In some embodiments, the applied magnetic field can be less than 10 T.

In preferred embodiments, a low-energy, static magnetic field is required to accomplish the delivery of MENCs across the BBB. In exemplary embodiments, approximately 0.8 T of magnetic field strength is needed to deliver MENCs to the brain of mice, while approximately 3 T of magnetic field strength is needed for the delivery of MENCs to the brain of a baboon.

The amount of drug released can be at least 10%, at least 20%, at least 25%, at least 30%, at least 40%, at least 50%, at least 60%, at least 70%, at least 75%, or at least 80% of the MENC's payload. The application of the magnetic field can be performed a second time to release a second amount of drug, with a desired length of time between applications of the magnetic field. This length of time can be, for example, at least 1 hour, at least 6 hours, or at least 12 hours separating the two applications of a magnetic field. The second application can be for the same amount of time as the first, or a different amount of time. It can be at the same field strength or a different (higher or lower) field strength as the first application, depending upon the amount of drug desired to be released at the second application. Whether after a single application or multiple applications, the amount of drug released to the subject can be, for example, at least 90%.

In an exemplary embodiment, an in-vitro non-invasive, magnetically guided delivery of MENCs comprising BaTiO₃@CoF₂O₄(BTO@CFO), 20-30 nm in size, to the brain is described as follows. Following dose optimization ranging from 5 mg/kg to 20 mg/kg based on cytotoxicity assays, an optimized nontoxic MENCs concentration of 10 mg/kg was injected into adult C57Bl/J mice. To ensure CNS delivery, MENCs injection was performed under the environment of a static magnetic field (0.8 T) for 3 hours. Results of the transmission electron microscopy (TEM) showed that MENCs were uniformly distributed in all cell population with minimal agglomeration. Further, organ-specific (brain, kidney, liver and spleen) and peripheral blood (hepatic and renal function test) toxicity were analyzed using standard Hematoxylin and Eosin (H&E) staining method and blood profiling method, respectively and showed no toxicity. Behavioral studies in MENCs injected mice were conducted to evaluate the effect of MENCs in neurocognition using specific neurobehavioral tests. Results of these studies showed no significant impairment in motor coordination when compared to untreated or PBS treated mice. The findings confirmed that MENCs can be delivered across the BBB and exhibit no apparent cytotoxicity or behavioral impairment in the mice, thus suggesting a potential application of MENCs for site-specific, on-demand and controlled delivery of therapeutics across the BBB to treat CNS diseases.

Advantages of using MENCs for targeted and on-demand drug delivery can be realized in at least the following four features.

First, methods provided herein allow one to control the electric-field bonding between the MENCs and drug molecules by magnetic fields instead of electric fields. Whereas the effects of electric fields are limited to the surface of the treatment area, magnetic fields can penetrate through the entire subject brain and be generated remotely. In addition, magnetic fields are less sensitive to static field and other noise sources.

Second, the use of magneto-electric materials enables efficient coupling between magnetic and electric fields. Remote magnetic fields are used to induce strong electric dipole charges (in MENCs) that can enhance or weaken the bond between the MENP carrier and the drug molecules. The bond, whether it is of ionic and/or covalent nature, is defined by its intrinsic electric fields, e.g. Coulomb forces. Therefore, each MENC serves as a nanoscale site that converts the magnetic energy of the remotely applied magnetic field to the electric energy needed to cause the release of the drug molecule from the MENC. This mechanism can provide at least 90% efficiency in the drug release process. For comparison, during conventional drug delivery and release processes where the bonding strength is controlled chemically, about 99% of the drug is lost as the nanoparticle carriers get deposited or eliminated through the reticuloendothelial system before it gets across the BBB.

Third, MENCs provided herein are preferably less than 50 nm in diameter, more preferably between about 20 nm and 30 nm, which is smaller than the BBB-defined size restriction, facilitating the efficient delivery of the drug molecules into the target treatment area in the subject brain.

Fourth, the approach provided herein is a non-invasive physical method for high-throughput delivery of MENCs and other magnetic particles. Among other currently available strategies, electroporation also relies on the use of electromagnetic forces. Unlike electroporation, the methods provided herein do not require creating any pores to mediate delivery. Thus, there is little chance of cellular damage, which is typical in electroporation of metal nanoparticles as heat generation is a concern (Bhardwaj, V., Srinivasan, S. & McGoron, A. J. Efficient intracellular delivery and improved biocompatibility of colloidal silver nanoparticles towards intracellular SERS immuno-sensing. Analyst 140, 3929 (2015)).

Animal subjects such as male C57Bl/J mice (e.g., EXAMPLES 2-11) of 6 weeks (n=6 for each experiment and each group) and baboons (e.g., EXAMPLE 12) were used in the following experiments. The mice were purchased from Jackson Laboratory and housed in standard ventilated cages with free access to water and food in a climate-controlled environment on a 12-h light/dark cycle (lights off at 09:00 h).

### Example 1

MENCs (BaTiO₃@CoFe₂O₄) synthesis is a 3-step process as described in previous publications (Nair, M. et al. S. Externally controlled on-demand release of anti-HIV drug using magneto-electric nanoparticles as carriers. Nat. Commun. 4, 1707 (2013); Guduru, R. et al. Magneto-electric nanoparticles to enable field-controlled high-specificity drug delivery to eradicate ovarian cancer cells. Sci. Rep. 3, 2953 (2013)). In brief, CoFe₂O₄ nanoparticles were prepared using a hydrothermal method. Step 1), 15 mL of aqueous mixture of 0.058 g of Co(NO₃)₂.6H₂O+0.16 g of Fe(NO₃)₃.9H₂O was combined with a second mixture of 0.2 g of polyvinylpyrrolidone (Average molecular weight was about 40,000 Dalton) dissolved in a 5 mL of aqueous solution with 0.9 g of sodium borohydride and heated at 120° C. for 12 hours. Step 2), the precursor solution of BaTiO₃ was prepared by mixing a 30 mL aqueous solution containing 0.029 g of BaCO₃ and 0.1 g of citric acid with a 30 mL ethanol solution containing 0.048 mL titanium isopropoxide and 1 g of citric acid. Steps 3), the BaTiO₃@CoFe₂O₄ MENCs were prepared by dispersing 0.1 g of CoFe₂O₄ nanoparticles in the precursor solution obtained in step 2. The suspension of both counterpart nanoparticles was sonicated for 2 hours. The well-dispersed mixture was dried at 60° C. overnight. Dried MENCs were allowed to calcinate at 780° C. for 5 hours. The average diameter of MENCs was controlled to be between about 20 nm and about 30 nm by controlling the cooling rate to be about 14° C./min.

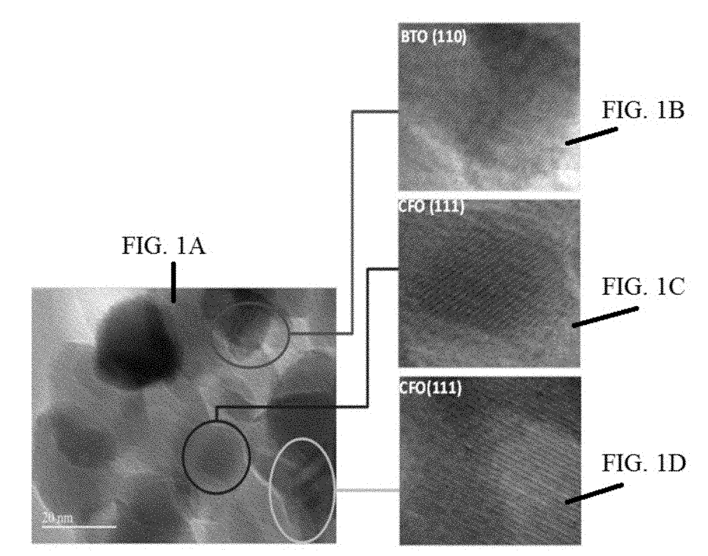

The particle size, distribution, morphology, and crystallinity of MENCs were studied using an FEI CM 200 transmission electron microscope (TEM). The particle size of the MENCs was estimated within 20 to 30 nm using TEM analysis. TEM results also confirmed that MENCs are composed of BTO and CFO phases (FIG. 1A), which is attributed to the appearance of atomic plains related to BTO and CFO (FIGS. 1B-1D).

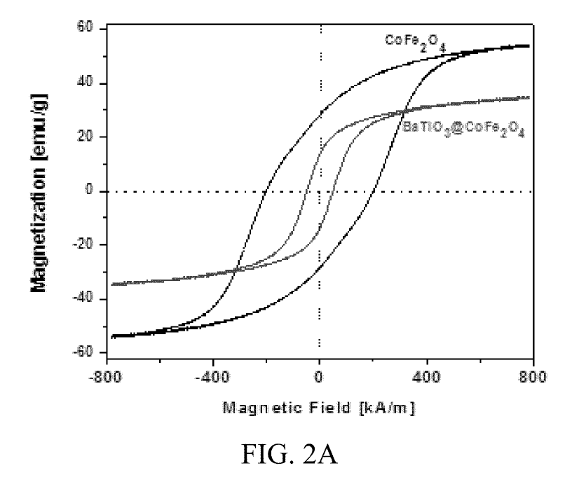

The phase composition of synthesized MENCs was studied using an X-ray diffractometer (based on Mo-Kα radiation) (FIG. 2B). Diffraction patterns of MENCs were analyzed and indexed using ICDD PDF 2014 database and Match software. The atomic interplanar spacing of CFO and BTO was estimated using Digital Micrograph Software. Observed peaks were indexed and attributed to both CFO (JCPDS 00-022-1086) and BTO (JCPDS 04-001-7269). Obtained diffraction peaks were broad due to the small size of MENCs. XRD pattern confirms that MENC synthesis resulted in CFO and BTO as the crystallographic planes. However, widths and intensities of the peaks are higher at some angles due to the overlapping of CFO and BTO planes.

The chemical fingerprint of the MENCs was studied using Raman Spectro-microscope (Nomadic Raman microscope with BaySpec 532 nm laser) (FIG. 2C). 20 μL of a 10 mg/mL aqueous solution of MENCs were drop-casted on silica substrate to acquire Raman spectra. Among several optically active Raman modes associated with BTO and CFO, the most intense modes characteristic to BTO and CFO were observed, respectively, around 500 cm⁻¹ due to dominant tetragonal phase, and 670 cm⁻¹ due to cubical inverse-spinel structure (Gajović, A. et al. Temperature—dependent Raman spectroscopy of BaTiO3 nanorods synthesized by using a template—assisted sol-gel procedure. J. Raman. Spectrosc. 44, 412 (2013)). BTO has 12 Raman modes as compared to CFO (5 modes), and therefore BTO dominates the Raman spectra. Due to the shift of Ti ions with respect to oxygen in BTO, the F₁ᵤ mode splits into A₁ and E modes, which are further separated into transversal (TO) and longitudinal (LO) components. BTO dominant peaks are observed at 300 cm⁻¹ due to E (TO+LO) mixed mode and peak at 515 cm⁻¹ due to E (TO) and A₁ (TO) transversal modes. The peak around 800 cm⁻¹ indicates stacking-fault density of BTO, which could be due to the high temperature (780° C.) used during MENC synthesis calcination. Five Raman active modes of CFO, one of A_{1g}, one of E_{g} and three of F_{2g} symmetries, are characteristic of the cubic inverse-spinel structure and in agreement with previous reports (Soler, M. et al. Structural stability study of cobalt ferrite-based nanoparticle using micro Raman spectroscopy. J. Magn. Magn. Mater. 272, 2357 (2004)). The F_{2g} symmetry, characterized by large oxygen motion and very small cobalt displacement, is observed around 670 cm⁻¹.

The polydispersity index (PDI) value of MENC was estimated as 0.22±0.03 using dynamic light scattering (DLS) method, suggesting that MENCs have good mono-dispersity in PBS. The hydrodynamic size of MENC was estimated at 90 nm, in agreement with the fact that the size measured with DLS (i.e. in aqueous form) should be higher than that measured with TEM (i.e. in dry form) at 20-30 nm due to the hydrophilic nature of the MENCs. The Zeta potentials of MENCs in PBS were estimated at −30 mV, suggesting a cationic surface of MENCs.

To evaluate the magnetism of the MENCs, magnetic hysteresis loops of the CoFe₂O₄ (CFO) and BTO@CFO at room temperature are presented in FIG. 2A. The obtained lower magnetization of MENCs (34 emu/g) compared to CFO (53 emu/g) was due to the deposit of BaTiO₃ (BTO) onto CFO surfaces and confirmed fabrication of MENCs. MENCs demonstrate ferromagnetic behavior that can be correlated with ferromagnetism and multiferroic, polarization properties of an electro-active material. This is a necessary phenomenon in order to achieve on-demand release through AC magnetic field stimulation via electromagnetic coils (Nair, M. et al. S. Externally controlled on-demand release of anti-HIV drug using magneto-electric nanoparticles as carriers. Nat. Commun. 4, 1707 (2013)).

### Example 2

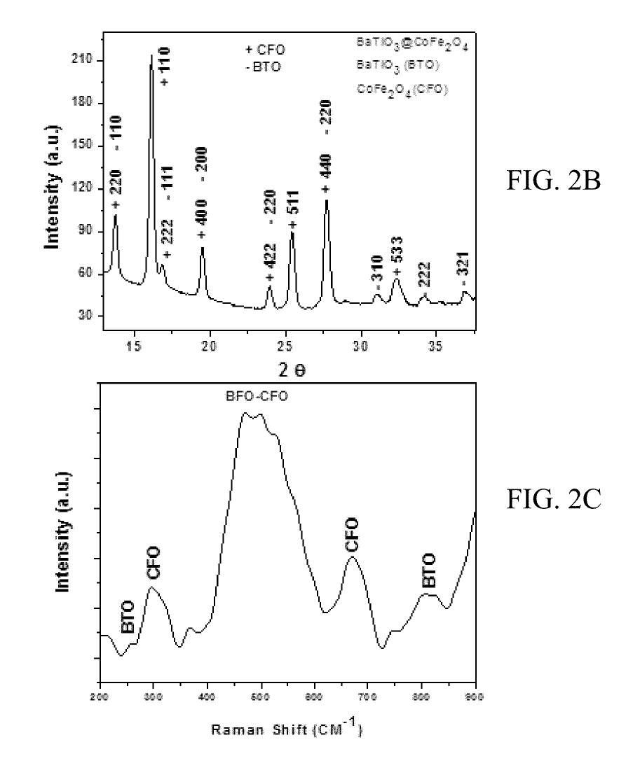

An MTT [3-(4, 5-Dimethylthiazol-2-yl)-2, 5-Diphenyltetrazolium Bromide] assay was used to study in-vitro cytotoxicity. Human astrocytes and SKNMC (1×106 cells/well) were grown in 6-well plates. Grown cells were treated with 100 μL of various MENCs doses (0.05-1 mg/mL). IACUC-approved doses were (5 to 20 mg/kg) for these experiments. For MTT assay, MENCs doses were back calculated with respect to the average mice weight of ˜20±1 gm as shown in FIGS. 3A-3B. Moreover, one dose less than 5 mg/kg and one higher than 20 mg/kg were also considered for MTT assay for better understanding.

Further, well plates were maintained in a humidifier incubator with an internal environmental consisting in 95% air and 5% CO2 at 37° C. After 48 days of incubation, one mL medium supplemented with 100 μL of MTT (100 mg MTT/20 mL PBS) was added to each well and incubated at 37° C. for 3 hours. Later, one volume of detergent reagent (20% SDS in 50% DMF) was added, rocked for about 2 hours, and then centrifuged. The optical density of the solubilized formazan was determined using UV-Visible spectrophotometer by measuring absorbance at 550 nm. The optical density of formazan in each well is directly proportional to the cell viability, utilized for calculations.

Results of MTT showed that treatments with different concentrations of MENCs exhibited high percentage of viability, more than 90% (for MENCs dose ranging from 0.05 to 0.25 mg/mL) in both cell lines, similar to the untreated control (FIGS. 3A-3B). However, the MENCs dose of more than 0.25 mg/mL showed lower cell viability ˜70%. The MENCs (5 to 15 mg/kg with respect to average mice weight 20±1) do not exhibit any cytotoxicity and are safe for in-vivo experiments. 10 mg/kg corresponding to 0.25 mg/mL (10 mg/kg) of MENCs exhibited a maximum of 96% cell viability for both astrocytes and neuronal cells and was selected for injection in C57Bl/J mice (FIG. 3C). An intermediate dose of 10 mg/kg was selected for in-vivo application due to easy detection and injection. Lower doses were difficult to detect and higher doses caused difficulty in injection due to high particle-particle interaction.

### Example 3

An optimized dose of MENCs (10 mg/kg with respect to 20±1 g mice) was used for injection in C57Bl/J mice (n=6/group, control n=6/group, males and 6 weeks old) in all experiments. (Charles River Laboratory, Inc., Wilmington, Mass.). MENCs were suspended in phosphate-buffered saline (PBS) in order to make an injection suspension.

A single dose (10 mg/kg) of MENCs was administered intravenously (i.v.-administration) in each mouse. Each mouse was sedated and its head placed in a stable external magnetic field (0.8 T) (FIG. 3C). The injected dose was selected to correspond to a human dose of 6.5 mg/kg to 20.3 mg/kg by interspecies allometric scaling factor.

After 3 hours of incubation, mice were kept at the normal cage condition under observation for a week. Intermittent blood samples were collected at days 2 and 7 in order to check Hematoxylin and eosin (H&E) staining and blood toxicity. The plasma supernatant was stored at −80° C. for analysis. Serum samples were analyzed for liver and renal panel toxicity.

After blood collection, mice were harvested to collect major organs such as brain, liver, kidneys, and spleen for histopathology. Histopathology analysis was done with H&E staining to observe any systemic toxicity in these tissues.

### Example 4

FEI CM 200 TEM was used for the morphological characterization of brain tissue of the control and the MENCs-injected brain tissue samples to evaluate MENCs transmigration, particle size distribution inside the brain and its uptake within the CNS cells. An in-vivo TEM study was also performed on PBS injected mice brain tissue using identical experimental conditions. Following the animal perfusion protocol (Tremblay, M.-E., Riad, M. & Majewska, A. Preparation of mouse brain tissue for immunoelectron microscopy. J. Vis. Exp. JoVE 41, e2021 (2010)), the mouse skull was chipped-off and tweezers were used to remove the brain and each of the two hemispheres was cut into 8 transverse blocks. The blocks were processed and analyzed for qualitative and quantitative uptake of MENCs using in-situ TEM and ICPMS, respectively. Protocols used to process brain tissue samples for these studies were adapted from a previous report (Bhardwaj, V., Srinivasan, S. & McGoron, A. J. Efficient intracellular delivery and improved biocompatibility of colloidal silver nanoparticles towards intracellular SERS immuno-sensing. Analyst 140, 3929 (2015)).

For in-situ TEM experiments, brain tissue blocks were cut into smaller sizes (˜50 μm thick), rinsed in ice-cold PBS three times, and fixed using 2% gluteraldehyde (primary fixative for 90 minutes) and 1% Osmium tetraoxide (secondary fixative for 30 minutes) with washing in between each individual fixation. Samples were transferred into watch glasses for serial dehydration, 35, 50, 70, 80, 90, 95 and 100%, using histology grade absolute alcohol for 20-30 minutes each. Dehydrated samples were embedded into Spurr's epoxy resin following manufacturer's guidelines, mixing components ERL, DER, NSA and DMAE in ratio 30:23:80:1. Samples were infiltrated using a series of resin:ethanol dilutions, 1:2, 1:1 and 3:1 and 100% resin for 3-5 hrs at each step. Sections were transferred into molds, filled with resin, and allowed to polymerize overnight at 70° C. in an Enviro-Genei incubator to obtain an isosceles trapezoid shape. Blocks were trimmed using a blade and cut into ultrathin sections (≦50 nm) using an ultra microtome (Porter-Blum MT-1, DuPont-Sorvall, USA) and a diamond knife (DDK, USA). Sections were collected in boats filled with acetone to help section stretching (ribbon-like) and were loaded on Ni grids with handles to allow ease of handling and robustness. The grids with samples were allowed to dry, observed under a light microscope to select the best samples, and stored in a grid box until TEM analysis.

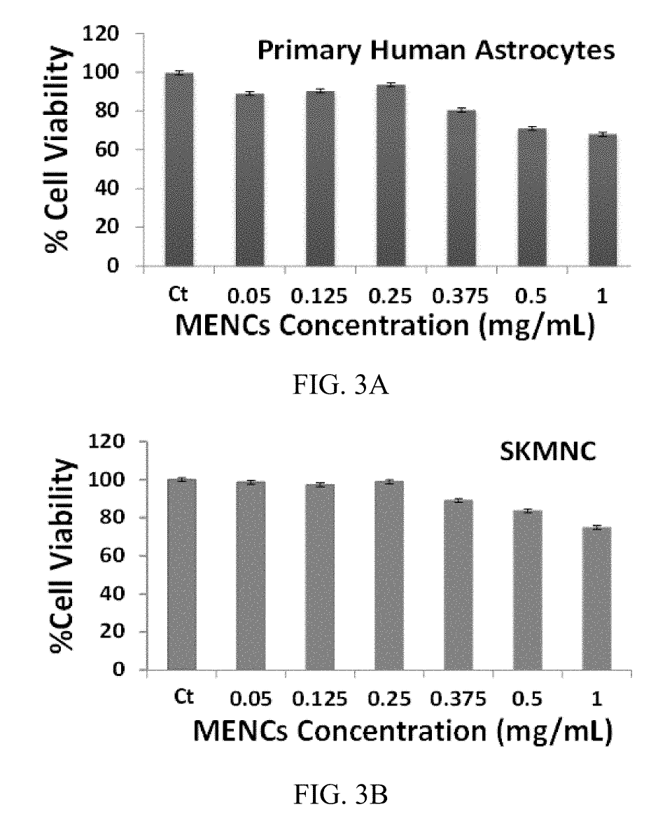

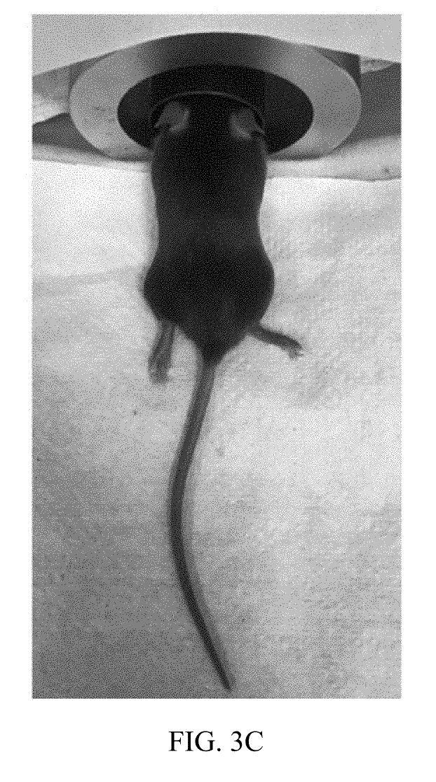

The qualitative uptake study using in-situ TEM imaging (FIGS. 4A-4B and 5A-5F) confirmed that MENCs were capable of efficiently crossing BBB (FIG. 4A vs FIG. 5A). Undoubtedly, abundant amounts of MENCs were found localized into brain cells, including neurons (FIG. 5A-5C), astrocytes and microglia. MENCs were also taken up by blood cells (FIG. 5A) and smooth muscle cells (FIG. 5E). Unlike TAT-mediated delivery across BBB, which resulted in endosomal entrapment of nanocarriers and failure to reach cell nuclei (Mikula, S., Binding, J. & Denk, W. Staining and embedding the whole mouse brain for electron microscopy. Nat. Methods. 9, 1198 (2012)), MENCs were found uniformly distributed inside cells and they were able to reach the nucleus in high numbers.

### Example 5

A quantitative estimation of MENCs across BBB was performed using inductively coupled plasma mass spectroscopy (ICP-MS). The ICP-MS studies were performed using Perkin Elmer Sciex, model ELAN DRC-II at the FIU Trace Elemental Analysis Facility. ICP RF power 1375, nebulizer gas flow 0.90 L/min, plasma gas flow 16 L/min, and lens voltage 8.25 V were selected as the acquisition parameter for each measurement. ICP-MS studies were performed to estimate Fe ion content in MENCs and to estimate MENCs concentration in MENCs injected mice brain.

Known concentrations of Ti (0, 10, 25, 50, 60, 80 and 100 ppb or μg/L) and Fe (0, 10, 25, 50, 60, 80 and 100 ppb or μg/L) in liquid suspension were used to establish calibration curves using ICP-MS. Ti (with respect to BTO) and Fe (with respect to CFO) were selected to confirm that MENCs do not lose chemical structure during CNS navigation. A good linearity was obtained for both elements with a regression coefficient (r²) of ˜0.998. ICP-MS study was conducted using a known concentration of MENCs to estimate the percentage of Fe content using a related calibration curve.

Results confirmed that MENCs consisted of 36.5% Fe content. Established calibration curves were also used to estimate the concentration of MENCs reaching mice brains. The brains of control mice were processed for ICP-MS study using identical conditions as reference. Results of the ICP-MS study confirmed that the concentration of Ti in tissue in the control and the MENCs-injected mice brains was 141 μg/g and 149 μg/g, respectively, and the concentration of Fe in tissue in the control and the MENCs-injected mice brains was 623 μg/g and 683 μg/g, respectively. Results confirmed that the concentration of MENCs in mice brains was 38 μg/g.

### Example 6

To establish references for estimating Fe ion concentrations of MENCs, calibration curves with respect to Ti and Fe ion were founded. MENCs suspended in PBS media were considered a blank negative control. Prior to ICP-MS analysis, samples were dissolved using the following acid digestion protocol: 1) Vortex the suspension and immediately place 20 μL of the suspended nanoparticles into a digestion polypropylene tube, and add 1000 μL of 16 M nitric acid (70%, optima grade), 2) Heat the samples in a dry heater block at 90° C. for 1 hour. Cover the vials with the digestion cap to avoid loss of volatile compounds. Check the samples to avoid complete dryness, 3) Remove the samples from the heater block and let them cool down at room temperature for several minutes, 4) Add 250 μL of hydrogen peroxide (30%, optima grade) and heat at 90° C. for 30 minutes, 5) Remove the samples from the heater block and let them cool down at room temperature for several minutes, 6) If needed, add 250 μL of hydrogen peroxide (30%, optima grade) and heat at 90° C. for 30 min until dry, 7) Remove the samples from the heater block and cool at room temperature, 8) Add 25 μL of Sc 10 ppm and dilute samples with nitric acid 0.8M to a final volume of 10 mL, followed by sonication for 15 minutes. Close the vial caps and vortex. This first dilution (1:500) is used to measure the content of Ti in the sample, 9), for analysis of Fe, a total dilution factor of 1:10000 is required. Reconstitute the dry sample to 10 mL with nitric acid 0.8M, then take an aliquot of 500 μL, add 25 μL of Sc 10 ppm and dilute to 10 mL. All samples were spiked with the internal standard (Sc). Each sample was analyzed in 6 to 9 replicates. Concentrations of Fe and Ti ions were in liquid suspension. A known concentration of MENCs (2 mg/mL) was used for ICP-MS study to measure Fe content in the formulation of MENPs.

### Example 7

Control and MENCs injected mice brain tissues were sliced and kept in a 1% formaldehyde solution. To remove water content, all samples were processed via serial dehydration using 24, 40, 60, 80, 100% ethanol. Prior to ICP-MS analysis the samples were digested using identical acid digestion process as used for MENCs Previously established calibration curves for Ti and Fe were used to estimate ion concentration in control and MENCs injected mice brain tissues.

For both experiments, optima grade nitric acid was used to prepare the calibration curve and standards. Three reagent blanks were exposed to the same digestion and dilution process as the rest of the samples. Quality control standards were prepared at concentrations of 25 ppb and 50 ppb, respectively, and analyzed at the beginning, middle and end of the analytical sequence. All quality control checks passed our quality control criteria (precision and bias better than 10%). Instrument blanks and reagent blanks were analyzed and the concentration values for the samples were then reported after their respective background subtraction.

### Example 8

In order to evaluate the chemical analysis of injected MENCs and their distribution inside the brain, FEI Tecnai F30 high-resolution transmission electron microscope (HRTEM) was employed in both TEM and STEM (Scanning Transmission Electron Microscopy) modes using the energy dispersive spectroscopy (EDS) technique. Selected area election diffraction (SAED) and convergent beam electron diffraction (CBED) patterns were obtained in TEM and STEM modes, respectively, to confirm the presence of the MENCs in the brain cells. The sampling for STEM study was similarly adapted to TEM studies.

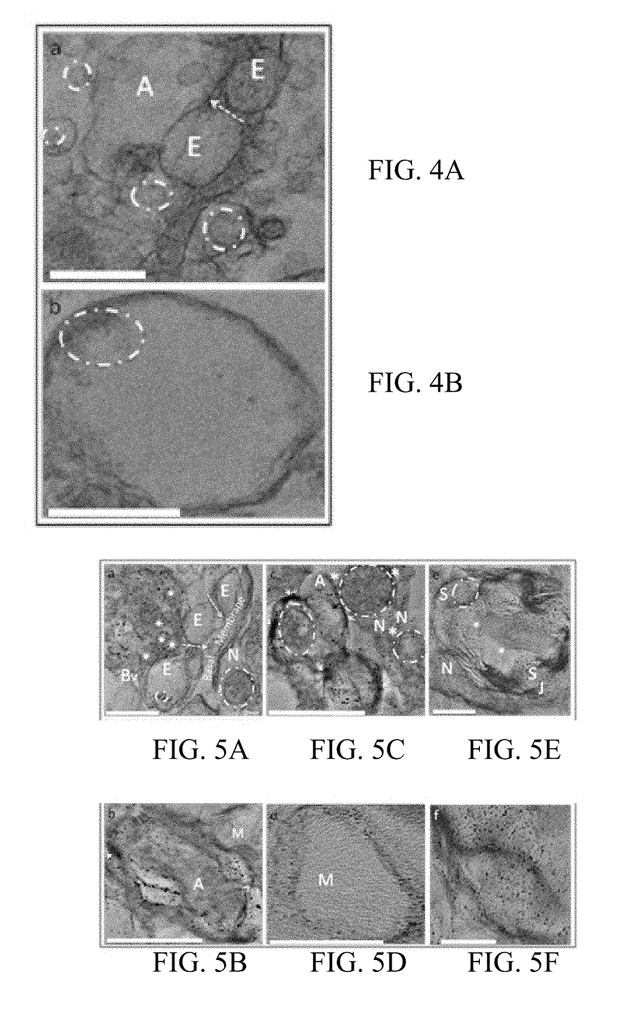

The ultrathin sections of mice brain, as used for in-situ TEM study, were subjected to scanning transmission electron microscopy (STEM) to evaluate elemental and structural analysis of the MENCs localized in the brain (FIGS. 6A-6K). STEM-based diffraction pattern was recorded at many locations (FIGS. 6A-6F) to explore crystalline integrity of MENCs in mice brain tissues (FIG. 6G). STEM images (FIGS. 6A-6F) corresponding to in-situ TEM (FIGS. 5A-5F) further confirms the presence of MENCs in BBB (FIG. 6A corresponding to FIG. 5A), astrocytes (FIG. 6B corresponding to FIG. 5B), Schwann cells surrounding axon terminal (FIG. 6C corresponding to FIG. 5C), periphery of neuron cells' nucleus (FIG. 6C corresponding to FIG. 5B), microglial cells (FIG. 6E corresponding to FIG. 5B) and cytosol of astrocytes (FIG. 6F corresponding to FIG. 5B). FIG. 6G shows the convergent beam electron diffraction (CBED) pattern of the MENCs in the brain cells in STEM mode to explore structural integrity. CBED pattern was indexed based on CFO (JCPDS 00-022-1086) and BTO (JCPDS 04-001-7269), and results confirmed the presence of MENCs inside the brain cells.

Brain tissues were also scanned at various positions and an identical energy-dispersive spectroscopy (EDS) spectrum was obtained for elemental analysis of the MENC. STEM images (FIGS. 6G-6I) depict MENC presence in arterioles and smooth muscle cells. A single spot from each image was selected for EDS analysis (FIG. 6K). The elemental analysis showed the presence of Ba, Ti, Co and Fe ions confirming that MENC does not lose its local chemical environment during transmigration across the intricate BBB. EDS was conducted in both TEM and STEM modes. The results were identical and indicated the presence of the Os, which was used for fixation and staining of mice tissue for in-situ TEM study, Ni from the TEM grid, C, O, Cl were primarily from the organic matter of the cell, and Co, Fe, Ba, and Ti from both CoFe₂O₄ (CFO) and BaTiO₃ (BTO). The obtained relatively very low intensity of Ba and Ti was due to merely a very thin layer/shell of BTO surrounding CFO core. The results of CBED and EDS confirmed that MENC does not disintegrate during the complex process of CNS delivery.

### Example 9

Tissue samples of brain, liver, kidney and spleen were fixed in 4% phosphate-buffered formaldehyde and paraffin-embedded according to conventional methods. For histopathological analysis, tissue sections were stained with hematoxylin and eosin (H&E). All systemic tissue toxicity was performed at the University of Miami's Department of Pathology. Histopathological evaluations were performed in accordance with the guidelines of the Society of Toxicologic Pathology. Images were taken using a Carl Zeiss Axio. All microscopic images were captured using an AxioCam MRc5 CCD camera.

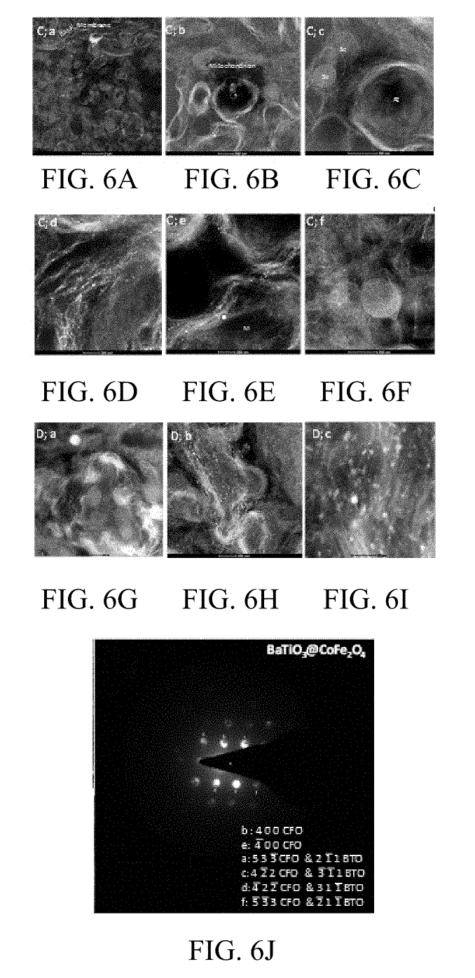

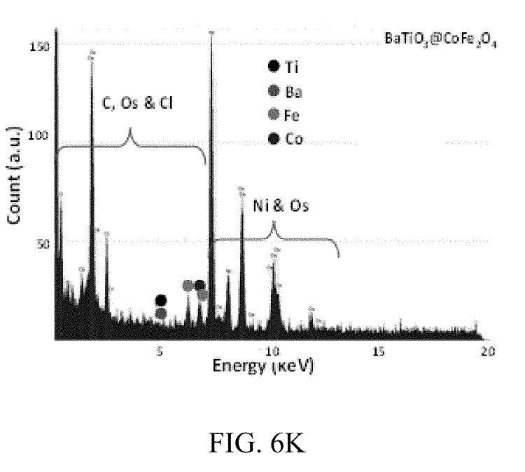

Histopathology analysis (FIGS. 7A-7D) showed that MENCs injected mice undergo minor extramedullary hematopoiesis as compared to control mice. In addition, H&E staining did not demonstrate any recruitment of macrophages or other immune cells in liver (FIG. 7B), kidney (FIG. 7A), spleen (FIG. 7C), and brain (FIG. 7D), indicating lack of toxicity. Results also confirmed that major organs of MENCs injected mice did not have any abnormality at cellular and organ levels. Moreover, the H&E staining of control and MENP-injected mice lung tissue did not exhibit toxicity similar to brain, liver, spleen, kidney and lung. This indicates no dramatic change happening during the process. Specific attention was given to the enzyme levels in order to best assess any evidence for induced toxicities. Blood biochemical tests (hemoglobin, creatinine, electrolytes, sodium, potassium, chloride, alanine aminotransferase, alkaline phosphatase, creatinine, total serum calcium and phosphate) and liver function tests (albumin, transaminases [alanine, aspartate and total bilirubin], gamma-glutamyl transferase and alkaline phosphatase) were performed; all values were found within normal reference range (FIG. 8). Overall the outcomes of above studies confirm that the delivery of MENCs is safe for both in-vitro and in-vivo systems.

For blood toxicity analysis (n=3 for both group of experiments), blood was collected using cardiac puncture at days 2 and 7 after the initial treatment. Serum chemistry profiles were analyzed through VRL, Maryland. The changes in enzyme levels were analyzed based on the normal range provided by VRL.

### Example 10

Based on the distribution of nanoparticles in different mouse brain regions, sensorimotor coordination was measured in mice injected with PBS, MENCs and no injection as the control group, using grip strength, horizontal bar and accelerating rotarod (Deacon) after days 2 and 7 post-treatment. Before testing, body weight was measured and each mouse was habituated to the environment for 20-30 minutes.

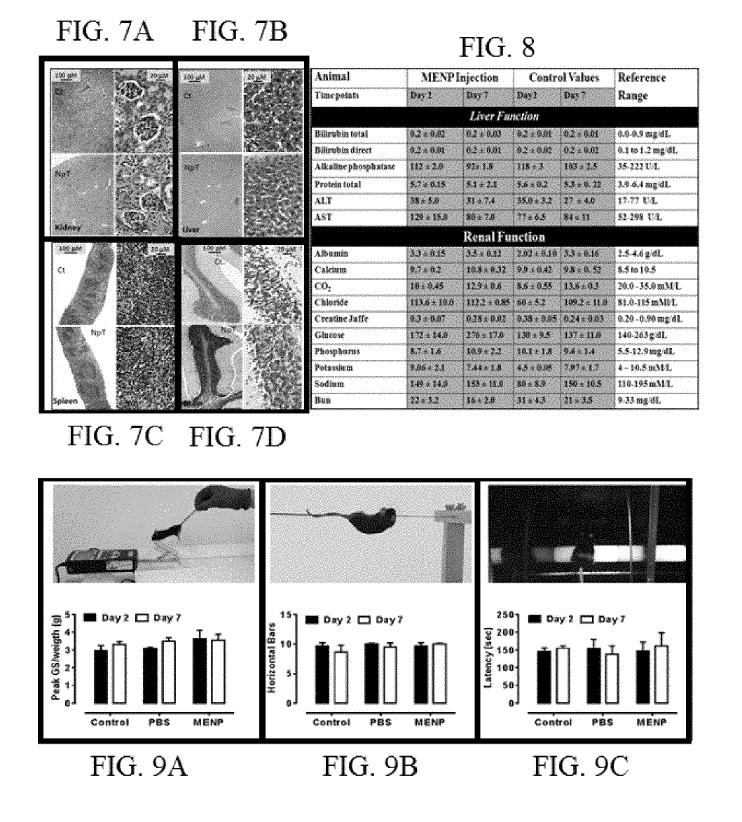

Grip strength allowed for the evaluation of motor weakness as this test relies on the instinctive tendency of the mouse to grasp an object with its forelimb. Grip strength was determined by placing each mouse with 2 limbs on a grid attached to a force gauge and steadily pulling the mouse by its tail. The grip force was automatically recorded using a computerized grip strength meter (SDI Grip Strength; San Diego Instruments, San Diego, Calif., USA). For each measurement, the test was performed in triplicate at 1 minute intervals, and an average of the values were taken and used as the animal's measure of grip strength. As shown in FIG. 9A, mice injected with MENCs (10 mg/kg) showed no difference, within the group, in forelimb grip strength after day 2 and day 7 of post-injection, when compared to PBS and no injection control groups.

Forelimb strength and coordination were further evaluated using 2 mm and 4 mm diameter horizontal bars, respectively. Mice were tested for their ability to hold onto the bar and/or touch the ends of the pole (supporting the bar). Falling after 30 seconds or placing one forepaw on a bar support without falling gave the mouse the highest score of 5. After the mice were scored for the 2 mm bar, it was then tested on the 4 mm bar, and the scores on the two bars were added. Forelimb grip strength was further tested using two horizontal bars and the combined scores from the 2 and 4 mm bars showed no apparent differences in test performances within groups or between the three groups at day 2 and day 7 (FIG. 9B).

An accelerated rotating rod test allowed us to evaluate coordination and motor skill acquisition (RotaRod-5; San Diego Instruments, San Diego, Calif., USA). Mice were placed on the rod without any training period and the rod was accelerated from 1 rpm to 40 rpm in 1.0 rpm step increment every 15 seconds. The time the mice spent on the rod without falling was recorded.

Furthermore, no differences in motor coordination were evident between the different treated animals, or when MENCs injected animals were compared to fall latency of mice injected with PBS and no injection controls at days 2 and 7 (FIG. 9C). Overall, presented results showed no impairment in motor performance between animals that were injected with MENCs, PBS or not injected control groups. The grip strength and rotarod test experiments using C57Bl/J mice are demonstrated in the videos.

### Example 12

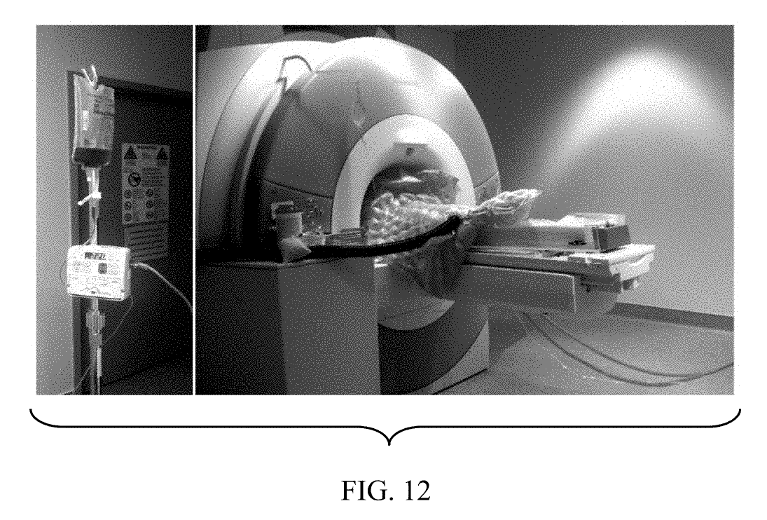

Baboon was also employed in an in-vivo experiment to examine the effectiveness of the site-specific, on-demand drug delivery method provided herein (FIG. 12).

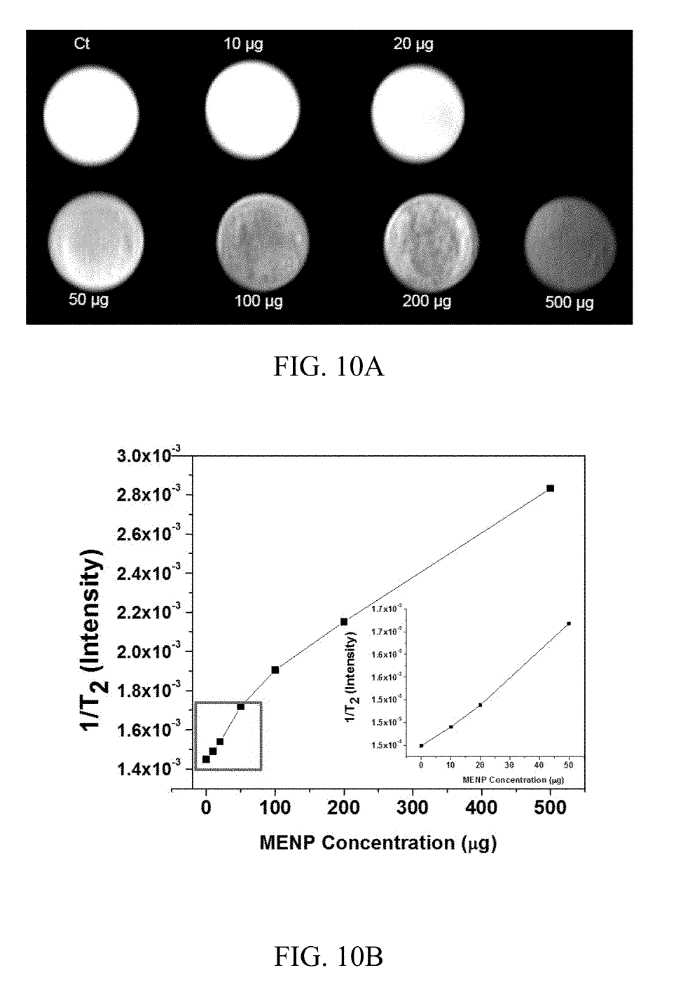

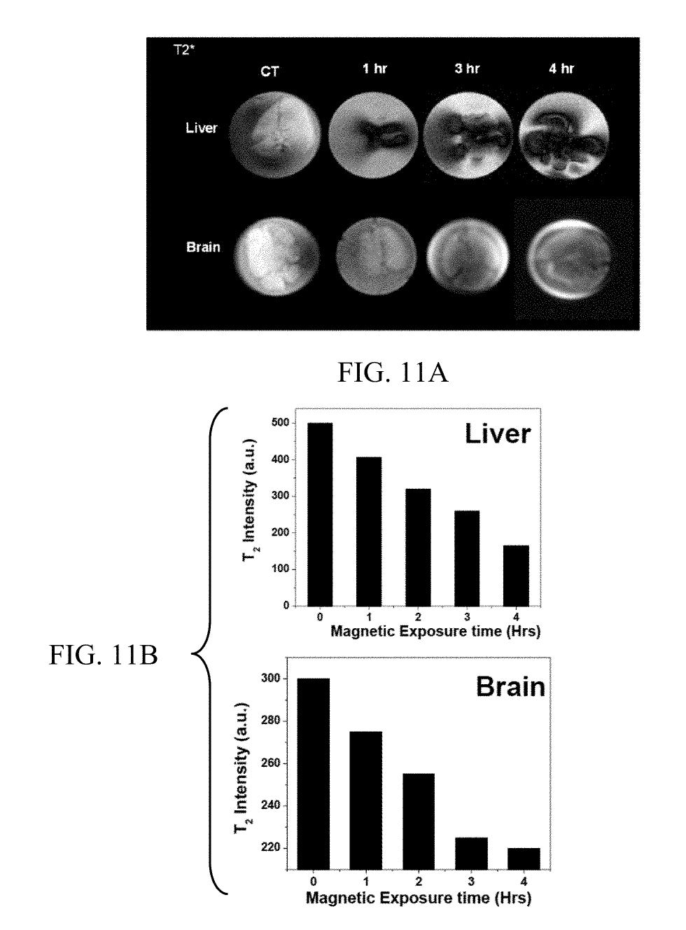

Specifically, in one set of experiments, a dose of 22 mg/13 kg MENCs (approximately 20 nm in diameter) suspended in 100 mL PBS with a flow rate of 220 mL/hr was injected in the baboon via Saphenous vein under static MRI magnetic treatment for 3 hrs. In another set of experiments, various dosages of MENCs (e.g., 10 μg, 20 μg, 50 μg, 100 μg, 200 μg, and 500 μg) were injected into a subject baboon (FIGS. 10A-10B) and the ex-vivo MRI analysis confirmed that 3 hours of exposure time achieved maximal CNS delivery (FIGS. 11A-11B).

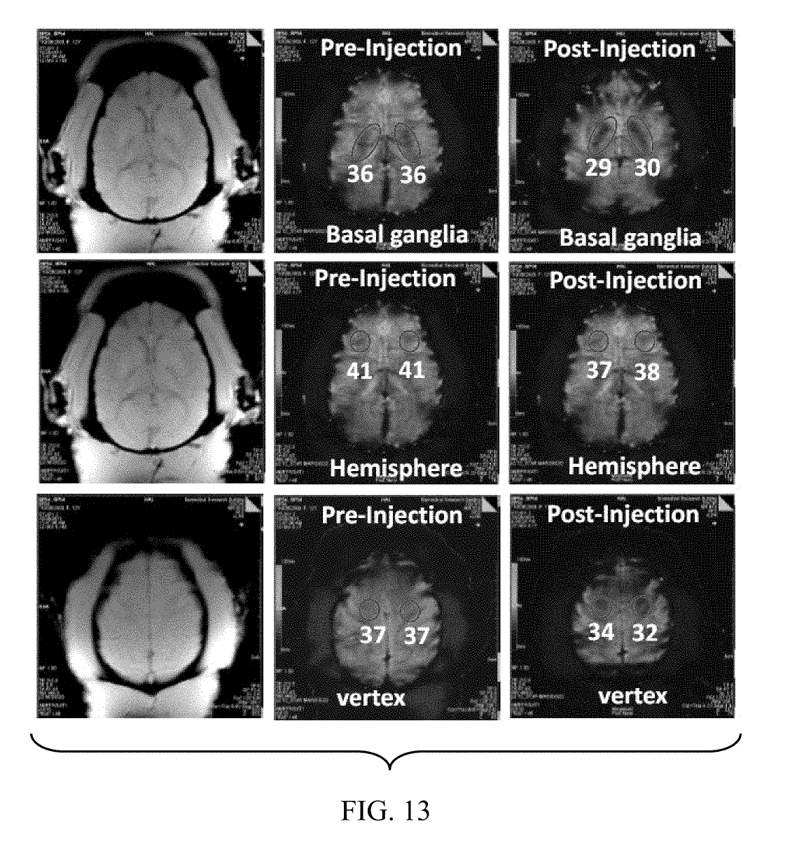

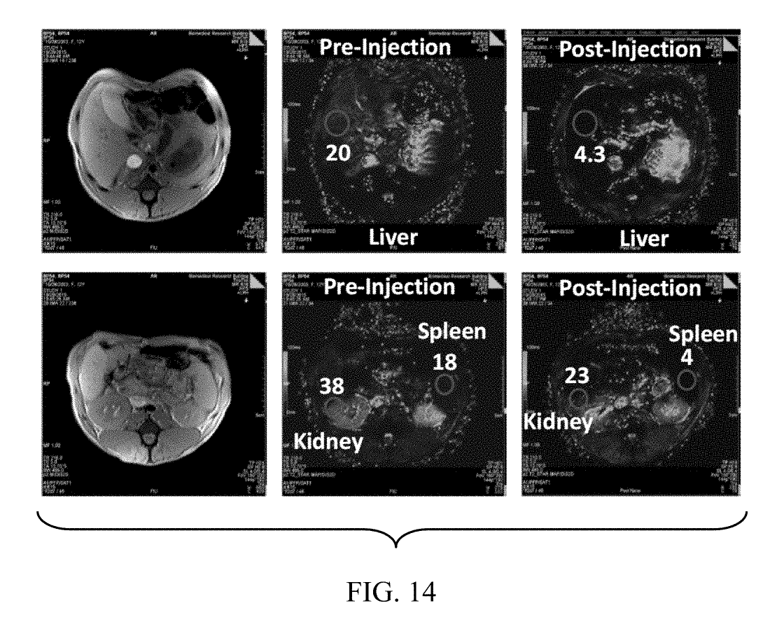

The results of the ensuing MRI-histopathological characterization showed MENCs transmigrate across the BBB (as seen in reduction in contrast in MRI images such as those shown in FIG. 13). In addition, abdominal MRI images also confirmed the presence of MENCs in peripheral system (FIG. 14).

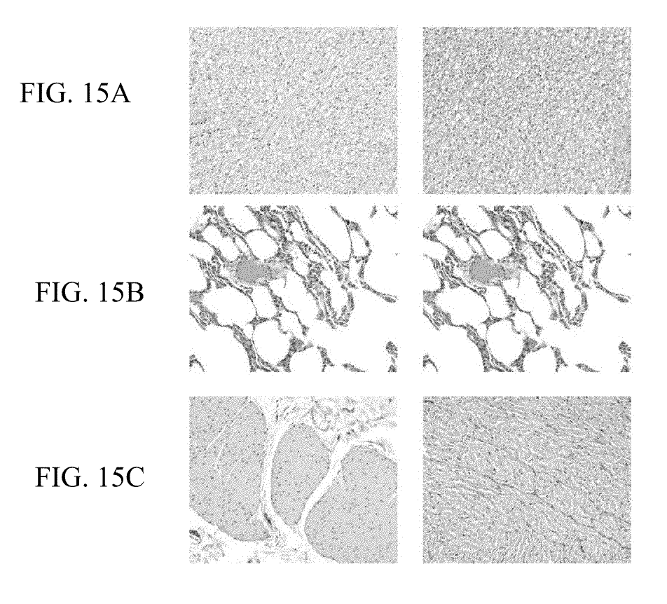

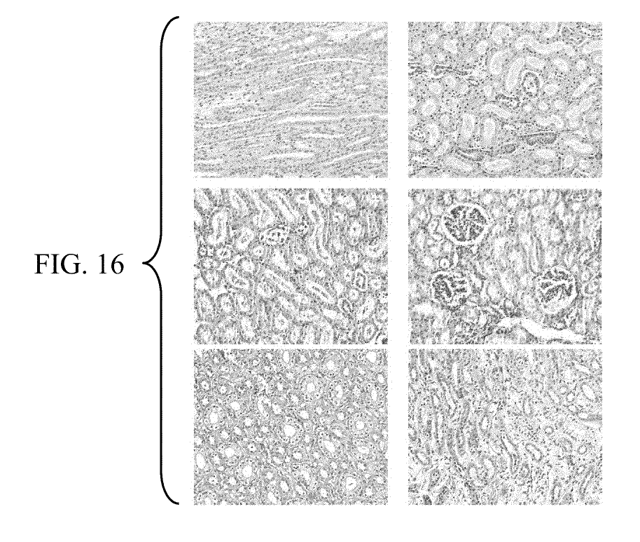

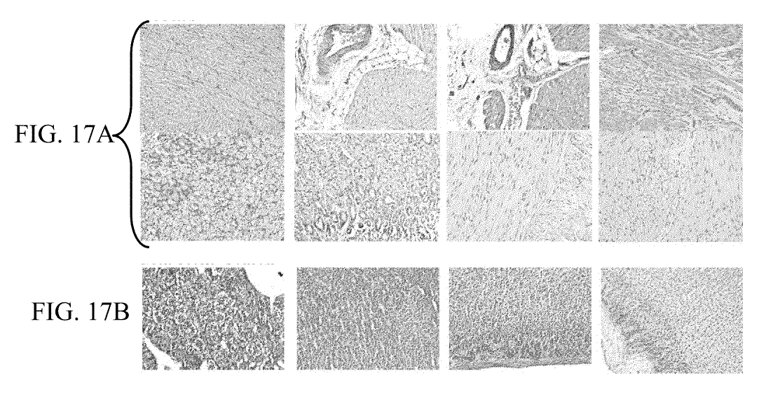

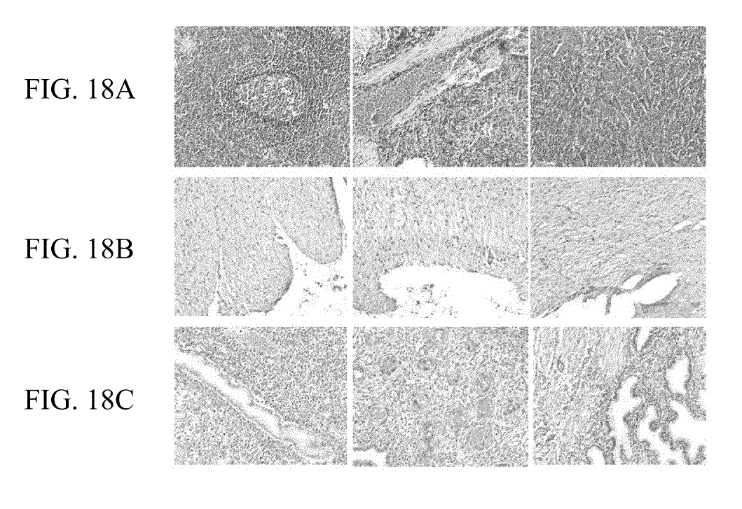

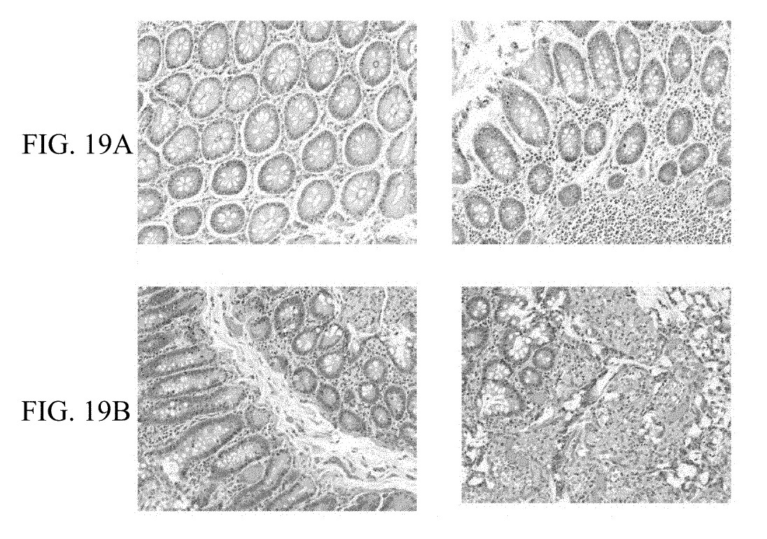

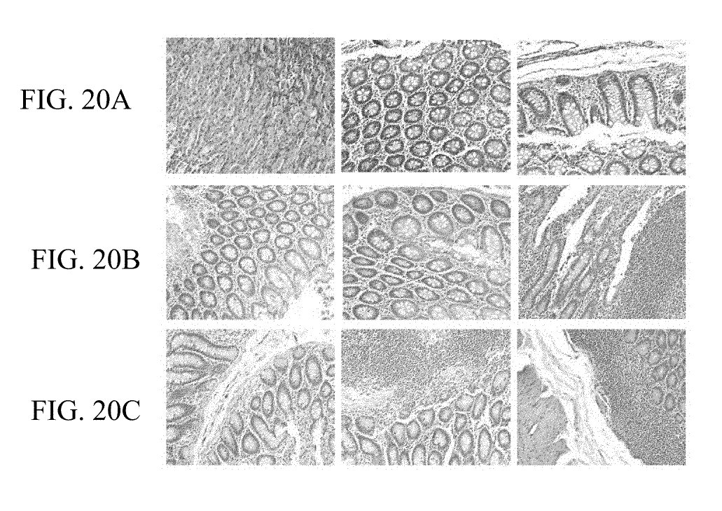

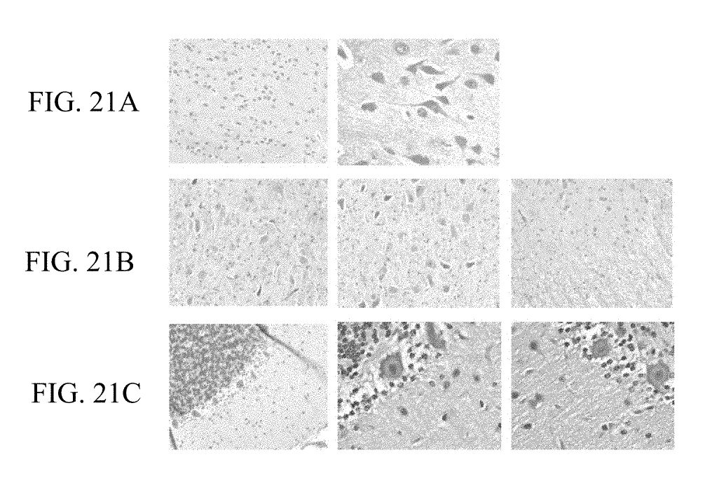

Tables 1 and 2 below demonstrate the blood toxicity profile of MENC-injected baboon 30 days following the treatment is within an acceptable physiological range. Furthermore, FIGS. 15-21 collectively demonstrate that the injection of MENCs into the subject baboon did not induce any organ-specific toxicity. Lastly, the subject baboon did not show any neurobehavioral or pathological side-effects, similar to the results of previous in-vitro and in-vivo studies involving mice.

TABLE 1
Blood toxicity profile of MENC-injected baboon (Part 1)

| | Physiological | | Post-injection | | | | |
|---|---|---|---|---|---|---|---|
| Parameters | Range | Pre-injection | Day 2 | Day 7 | Day 14 | Day 21 | Day 30 |

Blood toxicity profile

| | | | | | | | |
|---|---|---|---|---|---|---|---|
| WBC | 5.2 to 10.3 × 10³/μL | 8.0 | 6.9 | 8.2 | 11.5 | 5.2 | 8.3 |
| RBC | 5.2 to 6.0 × 10⁶/μL | 5.5 | 5.32 | 5.27 | 5.32 | 5.44 | 5.21 |
| Hemoglobin | 11.5 to 12.7 g/dL | 11.4 | 11.20 | 10.9 | 11.5 | 11.5 | 10.8 |
| Hematocrit | 35 to 39% | 37 | 38 | 37 | 37 | 37 | 36 |
| MCV | 65-70 fL | 67 | 71 | 70 | 70 | 67 | 69 |
| MCH | 21 to 23 pg | 21 | 21 | 21 | 22 | 21 | 21 |
| MCHC | 31 to 35% | 31 | 30 | 29 | 31 | 31 | 30 |
| Segmented | 30 to 57% | 76 | 68 | 62 | 74 | 67 | 73 |
| Neutrophils | | | | | | | |
| Band | 0 to 2% | 0 | 0 | 0 | 0 | 0 | 0 |
| Neutrophils | | | | | | | |
| Lymphocytes | 36 to 64% | 19 | 29 | 30 | 16 | 23 | 25 |
| Monocytes | 0 to 3% | 2 | 3 | 2 | 2 | 2 | 2 |
| Eosiniohils | 0 to 6% | 3 | 0 | 6 | 8 | 8 | 0 |
| Basophils | 0 to 1% | 0 | 0 | 0 | 0 | 0 | 0 |
| NRBC | | 0 | 0 | 0 | 0 | 0 | 0 |

| | |
|---|---|
| RBC | Normal |
| Morphology | |
| Platelet | Normal |
| Morphology | |
| WBC | Normal |
| morphology | |

TABLE 2
Blood toxicity profile of MENC-injected baboon (Part 2)

| | Physiological | Pre- | Pre-injection | | | | |
|---|---|---|---|---|---|---|---|
| Parameters | Range | injection | Day 2 | Day 7 | Day 14 | Day 21 | Day 30 |

Renal Function toxicity profile

| Hemolysis | 0 | 0 | | | | | |
|---|---|---|---|---|---|---|---|
| Index | | | | | | | |
| Lipemia Index | 0 | 0 | | | | | |
| Glucose | 47 to 65 mg/dL | 75 | 89 | 71 | 75 | 73 | 63 |
| BUN | 15 to 21 mg/dL | 18 | 13 | 15 | 15 | 11 | 29 |
| CREA | 0.5 to 0.9 mg/dL | 0.7 | 0.7 | 0.5 | 0.6 | 0.8 | 0.7 |
| BUN/CREA | 23 to 30 | 22.5 | 18.6 | 30.0 | 25.0 | 13.8 | 41.4 |
| Ratio | | | | | | | |
| Sodium | 142 to 150 mmol/L | 143 | 139 | 143 | 139 | 146 | 147 |
| Potassium | 3.3 to 3.8 mmol/L | 4.0 | 4.1 | 3.6 | 4.2 | 4.4 | 4.2 |
| Chloride | 106 to 115 mmol/L | 100 | 99 | 106 | 103 | 107 | 101 |
| CO₂ | | 28 mmol/L | 30 | 30 | 24 | 28 | 29 |
| Amylase | | 213 U/L | 214 | 213 | 255 | 150 | 237 |
| Calcium | 8.5 to 9.3 mg/dL | 9.1 | 9.6 | 8.8 | 9.1 | 9.9 | 10.5 |
| Phosphorus | 6.0 to 8.5 mg/dL | 4.4 | 2.4 | 3 | 3.6 | 5.1 | 1.5 |
| Cholesterol | 114 to 151 mg/dL | 99 | 88 | 98 | 89 | 135 | 88 |
| Total Protein | 6.8 to 7.5 g/dL | 6.1 | 6.0 | 5.8 | 5.7 | 6.9 | 6.7 |
| Albumin | 4.0 to 4.4 g/dL | 3.2 | 3.0 | 2.8 | 2.7 | 3.5 | 3.5 |
| A/G Ratio | 1.1 to 1.6 | 1.10 | 1.0 | 0.993 | 0.9 | 1.03 | 1.09 |
| AST | 19 to 48 U/L | 43 | 28 | 44 | 30 | 24 | 30 |
| ALT | 27 to 55 U/L | 51 | 62 | 60 | 44 | 51 | 53 |
| Alkaline | 89 to 470 U/L | 180 | 199 | 175 | 197 | 223 | 191 |
| Phosphatase | | | | | | | |
| Total Bilirubin | 0.1 to 0.5 mg/dL | <0.1 | <0.1 | 0.1 | <0.1 | 0.2 | 0.1 |

The examples and embodiments described herein are for illustrative purposes only and various modifications or changes in light thereof will be suggested to persons skilled in the art and are included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

All patents, patent applications, provisional applications, and publications referred to or cited herein are incorporated by reference in their entirety, including all figures and tables, to the extent they are not inconsistent with the explicit teachings of this specification.

### REFERENCES

1. Pardridge, W. M. Drug and gene targeting to the brain with molecular Trojan horses. Nat. Rev. Drug Dis. 1, 131 (2002).
2. Pardridge, W. M. Molecular Trojan horses for blood-brain barrier drug delivery. Current Opin. Pharmacol. 6, 494 (2006).
3. Pardridge, W. M. Blood-brain barrier delivery. Drug Dis. Today 12, 54 (2007).
4. Löscher, W. & Potschka, H. Drug resistance in brain diseases and the role of drug efflux transporters. Nat. Rev. Neurosci. 6, 591-602 (2005).
5. Nowacek, A. & Gendelman, H. E. NanoART, neuroAIDS and CNS drug delivery.

Nanomedicine 4, 557-574 (2009).
6. Lavan, D. A., McGuire, T. & Langer, R. Small-scale systems for in vivo drug delivery. Nat. Biotech. 21, 1184-1191 (2003).
7. Kaushik, A., Jayant, R. D., Sagar, V. & Nair, M. The potential of magneto-electric nanocarriers for drug delivery. Exp. Opin. Drug Del. 11, 1635 (2014).
8. Sagar, V. Pilakka-Kanthikeel, S. Pottathil, R. Saxena, S. K. & Nair, M. Towards nanomedicines for neuroAIDS. Rev. Med. Virol. 24, 103 (2014).
9. Mikula, S., Binding, J. & Denk, W. Staining and embedding the whole mouse brain for electron microscopy. Nat. Methods. 9, 1198 (2012).
10. Wegscheid, M. L., Morshed, R. A., Cheng, Y. & Lesniak, M. S. The art of attraction: applications of multifunctional magnetic nanomaterials for malignant glioma. Exp. Opin. Drug Del. 11, 957. (2014).
11. Cheng, Y., Morshed, R. A., Auffinger, B., Tobias, A. L. & Lesniak, M. S. Multifunctional nanoparticles for brain tumor imaging and therapy. Adv. Drug Del. Rev. 66, 42 (2014).
12. Cheng, Y. et al. Blood-Brain Barrier Permeable Gold Nanoparticles: An Efficient Delivery Platform for Enhanced Malignant Glioma Therapy and Imaging. Small 10, 5137 (2014).
13. Liu, L. et al. Polymeric micelles anchored with TAT for delivery of antibiotics across the blood-brain barrier. Pept. Sci. 90, 617 (2008).
14. Santra, S. Yang, H. Holloway, P. H. Stanley, J. T. & Mericle, R. A. Synthesis of water-dispersible fluorescent, radio-opaque, and paramagnetic CdS: Mn/ZnS quantum dots: a multifunctional probe for bioimaging. J. Am. Chem. Soc. 127, 1656 (2005).
15. Wu, Y.-C. et al. Massively parallel delivery of large cargo into mammalian cells with light pulses. Nat. Methods 0.12, 439 (2015).
16. Louboutin, J.-P. Chekmasova, A. A. Marusich, E. Chowdhury, J. R. & Strayer, D. S. Efficient CNS gene delivery by intravenous injection. Nat. Methods. 7, 905-907 (2010).
17. Bourzac, K. Nanotechnology: Carrying drugs. Nature 491, S58-S60 (2012).
18. Chen, Y. & Liu, L. Modern methods for delivery of drugs across the blood-brain barrier. Adv. Drug Del. Rev. 64, 640 (2012)
19. Jayant, R. D. et al. Sustained-release nanoART formulation for the treatment of neuroAIDS. Int. J. Nanomed. 10, 1077 (2015).
20. Nair, M. et al. S. Externally controlled on-demand release of anti-HIV drug using magneto-electric nanoparticles as carriers. Nat. Commun. 4, 1707 (2013).
21. Guduru, R. et al. Magneto-electric nanoparticles to enable field-controlled high-specificity drug delivery to eradicate ovarian cancer cells. Sci. Rep. 3, 2953 (2013).
22. Gajović, A. et al. Temperature—dependent Raman spectroscopy of BaTiO3 nanorods synthesized by using a template—assisted sol-gel procedure. J. Raman. Spectrosc. 44, 412 (2013).
23. Soler, M. et al. Structural stability study of cobalt ferrite-based nanoparticle using micro Raman spectroscopy. J. Magn. Magn. Mater. 272, 2357 (2004).
24. Bhardwaj, V., Srinivasan, S. & McGoron, A. J. Efficient intracellular delivery and improved biocompatibility of colloidal silver nanoparticles towards intracellular SERS immuno-sensing. Analyst 140, 3929 (2015).
25. Tremblay, M.-E., Riad, M. & Majewska, A. Preparation of mouse brain tissue for immunoelectron microscopy. J. Vis. Exp. JoVE 41, e2021 (2010).
26. Khizroev, S., Nair, M. P. N., Liang P., Runowicz, C., Gururu, R. On-demand drug release using magneto-electric nanoparticles, WO 2014/074584 A1.
27. Khizroev, S., Nair, M. P. N. Method for non-invasive brain stimulation, U.S. Pat. No. 9,272,158 B2.

We claim:

1. A method of delivering a drug to the brain of a subject, comprising:
administering to the subject a composition comprising magneto-electro nanocarriers (MENCs) loaded with the drug, and
applying a static magnetic field directed toward a treatment area of the brain, the strength of the magnetic field being sufficient to deliver the MENCs across a blood brain barrier (BBB) to the treatment area.

2. The method according to claim 1, the composition comprising MENCs comprising a multiferroic material such that an application of an external magnetic field induces a change in the electric dipole moment within each MENC.

3. The method according to claim 2, the composition comprising MENCs having a CoFe₂O₄ core and a BaTiO₃ shell surrounding the core.

4. The method according to claim 1, the subject being a human or a non-human mammal.

5. The method according to claim 4, the subject being a mouse or a baboon.

6. The method according to claim 1, the drug being capable of treating a central nervous system disease selected from brain tumors, neuroAIDS, and neurodegenerative diseases.

7. The method according to claim 1, the composition comprising MENCs having a diameter between 20 nm and 30 nm.

8. The method according to claim 1, the MENCs being administered at a dosage of between 5 mg/kg and 15 mg/kg.

9. The method according to claim 1, the strength of the static magnetic field being less than 10 T.

10. A method of treating the brain of a subject, comprising:
administering a composition comprising magneto-electro nanoparticles (MENCs) loaded with a drug, each MENC having a diameter of less than 50 nm;
applying a first magnetic field directed toward a treatment area of the brain, the strength of the first magnetic field being sufficient to deliver the MENCs across a blood brain barrier (BBB) to the treatment area; and
applying a second magnetic field directed toward the treatment area of the brain, the strength of the second magnetic field being sufficient to release the drug in a controlled manner,
the first magnetic field being a static magnetic field and the second magnetic field being an alternating current magnetic field.

11. The method according to claim 10, the composition comprising MENCs having a CoFe₂O₄ core and a BaTiO₃ shell surrounding the core.

12. The method according to claim 10, the subject being a human or a non-human mammal.

13. The method according to claim 12, the subject being a mouse or a baboon.

14. The method according to claim 10, the drug being capable of treating a central nervous system disease selected from brain tumors, neuroAIDS, and neurodegenerative diseases.

15. The method according to claim 10, the composition comprising MENCs having a diameter between 20 nm and 30 nm.

16. The method according to claim 11, the MENCs being administered at a dosage between 5 mg/kg and 15 mg/kg.

17. The method according to claim 11, the second magnetic field comprising an alternating-current magnetic field having a strength of less than 10 T.

18. A method of treating the brain of a subject, comprising:
injecting the subject with an aqueous solution comprising magneto-electro nanoparticles (MENCs), the MENCs having a diameter of between 20 nm and 30 nm and comprising CoFe₂O₄ at the core and BaTiO₃ at the surface of each nanoparticle, and the MENCs being loaded with a drug capable of treating a brain disease; and
applying a static magnetic field, having a strength of less than 10 T, directed toward a treatment area of the brain, the strength of the magnetic field being sufficient to deliver the MENCs across the blood brain barrier (BBB) to the treatment area.

19. The method according to claim 18, the subject being a mouse and the strength of the static magnetic field being less than 1 T.

20. The method according to claim 18, the subject being a baboon and the strength of the static magnetic field being less than 5 T.
